(19) United States
(12) Patent Application Publication
(10) Pub. No.: US 2022/0175255 A1
(43) Pub. Date: Jun. 9, 2022

(54) A SYSTEM AND A METHOD FOR MEASURING VOLUMETRIC FLOW IN ARTERIES

(71) Applicant: Rigshospitalet, Copenhagen Ø (DK)

(72) Inventor: Muhammad SABBAH, Kgs. Lyngby (DK)

(21) Appl. No.: 17/598,933

(22) PCT Filed: Mar. 31, 2020
(86) PCT No.: PCT/EP2020/059106

§ 371 Date: Sep. 28, 2021

(30) Foreign Application Priority Data
Apr. 1, 2019 (DK) PA 2019 70205

(51) Int. Cl. A61B 5/026; A61M 25/01; A61M 25/00; A61B 5/00

(52) U.S. Cl. CPC A61B 5/026; A61M 25/01; A61M 2025/0035; A61M 25/0068; A61B 5/6853; A61M 25/0026

(57) ABSTRACT

An arterial flow measurement system comprising: a catheter tube being which may be configured to be inserted into an artery, the catheter tube having longitudinal axis, a proximal end and a distal end, a first inner lumen providing fluid communication along the longitudinal axis of the catheter tube, a first opening (input opening) configured to provide fluid communication from the outside of the catheter tube and into the first inner lumen through a side wall of the catheter tube, a fluid guide element having an insertion state where the fluid guide element has a first cross-sectional diameter, and in a measuring state where the fluid guide element has a second-cross-sectional diameter, where the fluid guide element is configured to guide at least part of an arterial flow surrounding the fluid guide element into the first opening, a conversion element, where the conversion element is connected to the fluid guide element allowing a user to selectively transform the fluid guide element from an insertion state to a measuring state, and vice versa; and where the arterial flow measurement system comprises a flowmeter configured to measure the flow of a fluid wherein the flowmeter is in fluid communication with the first inner lumen of the catheter tube

The present disclosure relates to an arterial flow measurement system comprising a catheter tube and related methods.

### BACKGROUND

In cardiovascular medicine, a number of methods exist, where the methods involve certain measurements whose purpose is to evaluate or quantify the degree of underlying disease in order to assist medical professionals in making a treatment decision. One of the challenging things to measure directly in the human body is volumetric blood flow, as it is complicated by, among other things, i) nonlinear fluctuations in driving pressure ii) pulsatile flow iii) compliant blood vessels iv) flow of non-newtonian fluid (blood) v) difficulty in gaining physical access to blood vessels. The vitality and function of all tissues—particularly heart muscle—depend on blood flow. Therefore, information regarding blood flow is of outmost interest to the medical professional.

There are several ways of measuring physical parameters inside arterial and venous blood vessels. Wires or catheters can be inserted in blood vessels, with the wires or catheters containing various sensors e.g. pressure transducers, thermistors and pizo-electric crystals which can measure the parameters of interest. Materials can be introduced into the vessels and be used to follow the flow of blood using various imaging technologies such as x-rays, magnetic resonance or positron emission tomography.

Coronary Flow Reserve (CFR) is defined as the ratio of maximal hyperaemic blood flow to resting blood flow in the coronary arteries. Maximal blood flow can be induced by infusion of substances in either the coronary arteries or in peripheral blood vessels in the body. These substances, e.g. adenosine, relax the coronary artery walls, and thereby expands the coronary artery lumen in the plane perpendicular to the artery's longitudinal axis, i.e. it increases the artery's diameter. The increase in artery diameter in turn lowers hydrodynamic resistance and thereby facilitates increased flow, i.e. maximal hyperaemic blood flow. Thus, CFR represents the magnitude of increase in blood flow that the circulation can produce from a set-point that is defined as resting blood flow. Thus, a high CFR represents a great ability to increase blood flow from the resting value, whereas a low CFR represents a poor ability to increase blood flow from the resting value. CFR is used to evaluate the health condition of the entire coronary circulation. CFR is also used to evaluate the efficacy of treatments targeted at the coronary circulation. It is important to note that, from a medical perspective, it is more important to obtain information about the ratio of maximal hyperaemic blood flow to resting blood flow, rather than the absolute value of either. Therefore, any method that can provide an index of maximal hyperaemic to resting blood flow can be counted as expressing CFR. Thus, measuring CFR does not necessarily depend on directly or indirectly measuring absolute maximal hypermaemic blood flow and absolute resting blood flow, and dividing the two. However, due to the aforementioned complex non-linearities of blood flow, and in particular coronary blood flow, any deviations from a CFR calculation based on direct measurement of volumetric blood flow must necessarily involve several assumptions and approximations.

The known methods for measuring the blood flow through the arteries, are methods that do not measure blood flow, but are at best estimates of blood flow through the arteries, where such methods are e.g. intracoronary thermodilution of tempered saline, intracoronary doppler flow, doppler echocardiography, and positron emission tomography. None of these methods are capable of measuring volumetric blood flow in the arteries directly, but rather use surrogate measures such as thermodilution curves, instantaneous blood velocity and positron emission from the flowing blood, to calculate or estimate blood flow in the arteries. All these indirect methods use numerous assumptions and are subject to several methodological sources of error as well as errors related to heterogenous physical attributes of patients.

Thus, there is a need for an improved method that allows for measurement of blood flow in the arteries based on a direct measurement of volumetric blood flow, and not a surrogate measure of blood flow. The benefit of direct measurement of volumetric blood flow will be a method with fewer assumptions, a robust theoretical framework and a consistent and easy method of measurement.

### GENERAL DESCRIPTION

In accordance with the present description, there is provided an arterial flow measurement system comprising: a catheter tube being which may be configured to be inserted into an artery, the catheter tube having longitudinal axis, a proximal end and a distal end, a first inner lumen providing fluid communication along the longitudinal axis of the catheter tube, a first opening (input opening) configured to provide fluid communication from the outside of the catheter tube and into the first inner lumen through a side wall of the catheter tube, a fluid guide element having an insertion state where the fluid guide element has a first cross-sectional diameter, and in a measuring state where the fluid guide element has a second-cross-sectional diameter, where the fluid guide element is configured to guide at least part of an arterial flow surrounding the fluid guide element into the first opening, a conversion element, where the conversion element is connected to the fluid guide element allowing a user to selectively transform the fluid guide element from an insertion state to a measuring state, and vice versa; and where the arterial flow measurement system comprises a flowmeter configured to measure the flow of a fluid (liquid), wherein the flowmeter is in fluid communication with the first inner lumen of the catheter tube.

Within the context of the present disclosure the term proximal and distal may be seen as relative terms, where in this disclosure the term proximal relates to the relative position in relation to the body of the user. Thus, the term proximal end means the end closer to the user in the direction of insertion of the catheter, while the term distal end means the end that faces away from the user in the direction of insertion of the catheter.

The blood flow in the arteries of a body is a pulsatile flow, where the flow has periodic variations which relate to the pumping actions of the heart. The increase in flow may cause an increase in pressure in the arteries, as the pumping action forces the blood into a predefined volume of arteries. Even though the blood pressure may be representative of a an increase in flow of blood, the correlation between the increase in flow and the increase in pressure cannot be seen as being directly correlated, as the pressure increase is dependent on numerous physical attributes in the body, such as the compliancy of the blood vessels, and how the blood vessels can expand during the increase pressure. It is a known fact that a plurality of physical conditions can affect the compliancy of the blood vessels, which means that the compliancy of the blood vessels for one person are not necessarily the same as for another person. Thus, any measurements of blood flow that are dependent on the pressure of the blood may be seen as having an unknown reliability and measurement accuracy, as the blood pressure is dependent on a plurality of factors that do not relate to the blood flow.

In accordance with the present description, the catheter may be intended to be introduced into an artery, where the catheter may e.g. be intended to be introduced into an artery that is distal to the heart, i.e. the femoral artery, radial artery, internal jugular vein and/or other suitable veins and/or arteries, where the catheter may be threaded into its correct position using imaging technologies to guide the catheter in its correct position.

When the catheter is introduced, it may be in its insertion state, where the guide element may be in a state where the guide element does not impede the guiding of the catheter into its position. When the catheter is in is correct position, the guide element may be transitioned into its measurement state, where the fluid guide element may capture a part of the arterial flow, and provide a pathway for the arterial flow into the first opening, allowing a part of the arterial flow (blood) to be introduced into a proximal end of the catheter and to feed it out of the catheter for feeding it into a flowmeter, to measure the flow of the blood that has been diverted out of the artery.

The arterial flow through the artery is the blood flow through the artery, and when the catheter is inserted into the correct position, and the fluid guide element has been transitioned into its measurement state, at least a part of the blood flow may be diverted from the artery and into the catheter, so that the flow through the catheter may have a direct correlation to the blood flow through the artery. The blood may be diverted into the first opening, where the first opening provides access to a first inner lumen of the catheter, and where the blood flow may e.g. be used to push the blood from the first opening and towards the flowmeter, where the flowmeter registers the flow of blood through the catheter. As there is a correlation between the flow of blood through the catheter and the flow of blood through the artery, the flow result originating from the flowmeter may be used to derive and/or calculate the blood flow in the artery.

The blood fluid guide and/or the first opening and/or the lumen may be configured to guide a predetermined part of the flow towards the flowmeter, so that the flow measured may be seen as being representative of the flow occurring inside the artery. The fluid guide may be formed in such a manner that a part of the blood flow through the artery is diverted or guided towards the first opening, and where the blood enters the first opening, the blood is fed onwards towards the flowmeter. This means that the flowmeter is capable of measuring the actual flow of blood through the catheter, and where the actual flow of blood through the catheter is therefore correlated to the flow through the arteries. Thus, the measurement in the flowmeter may e.g. correspond or be comparable at least in part with the actual blood flow in the artery. Any variations in the blood flow, such as resistance in the flow of blood from the fluid guide and towards the flowmeter, that is fed into the catheter may be calculated and/or measured, so that any drop in flow may be anticipated and/or calibrated into the system and taken into account when the blood flow through the arteries is evaluated from the measurement from the flowmeter.

The conversion element of the catheter may be connected to the fluid guide element, so that the conversion element may be operated by a healthcare professional in such a manner, so that when the catheter is in its correct position, the fluid guide may be transformed from its insertion state to its measurement state, so that the blood may be directed towards the first opening. The conversion element may be a mechanical element that is implemented in the catheter, so that the fluid guide element may be manipulated from a distance, where one end of the conversion element is connected to the fluid guide element, while an opposing end of the conversion element may be connected to a control member, allowing the conversion element to be manipulated. The conversion element may e.g. be a resilient member, that is adapted to transform the fluid guide element when it is in position via e.g. external manipulation, release of a locking member infusion of a fluid and/or liquid, or similar means. The fluid may be saline that is infused with a contrast medium for X-ray imagery.

The first and/or second cross-sectional diameter of the fluid guide element may be seen in a predefined section of the catheter, where the cross-sectional diameter may be seen in a radial direction of the catheter, i.e. in a direction that is perpendicular to the longitudinal axis of the catheter. Thus, the cross-sectional diameter may be seen as the width of the fluid guide. The first and/or second cross-sectional diameter may be equal to or larger than the cross-sectional diameter of the catheter tube, so that the fluid guide may be adapted to intercept or interrupt the flow of blood that abuts the catheter tube. The first and/or second cross-sectional diameter may be adapted to be smaller than the cross-sectional diameter of the artery where the measurement is to be made, so that at least part of the blood flow is capable of passing the fluid guide element, when it is in its measurement state.

During use, the catheter may be configured or intended positioned in such a way inside the artery that the direction of flow of the blood is parallel to the longitudinal axis of the catheter, and where the direction of flow is in the direction from the distal end towards the proximal end of the catheter.

The flowmeter may be an in vitro flowmeter, where the flowmeter is outside the body of the user, and where the flowmeter is in fluid communication with the first lumen of the catheter.

The first opening may be in fluid communication with the flowmeter. This means that the blood flow may be directed via a fluid communication pathway from the first opening towards the flowmeter. The fluid communication pathway may be a closed fluid communication pathway.

In one or more exemplary embodiments, the fluid guide element may be positioned between a proximal tip of the catheter and the first opening. The proximal tip of the catheter may be a tip that closes off one or more lumens inside the catheter, and where the proximal tip may be rounded in order to reduce the risk that the tip of the catheter may damage the arteries and/or the veins during insertion of the catheter into the body. The first opening may be positioned at a predetermined distance from the proximal tip, where the fluid guide element may be positioned in an area of the catheter that is between the proximal tip and the longitudinal position of the first opening. This means that the flow of blood in the artery may be adapted to flow along the outer surface of the catheter, where the blood may pass the first opening, and subsequently the blood will intersect the flow guide. Thus the flow guide may be adapted to guide a part of the blood flow in a direction that is different from the flow of blood through the artery, and where the opening may be prior to the guide element, a part of the blood flow may be forced in a direction that is opposed to the direction of flow through the artery. Thus, the fluid guide element may be adapted to force the flow of blood into the opening and the lumen in a direction that is opposite to the flow of blood inside the artery. I.e. the guide element may be configured to intercept and change the flow direction of at least part of the blood flow inside the artery.

In one or more exemplary embodiments, the catheter, system and the method in accordance with the present disclosure is configured to measure Coronary Flow Reserve in a human body.

In one or more exemplary embodiments, the first cross-sectional diameter may be smaller than the second cross-sectional diameter. The first cross-sectional diameter of the flow guide element may be adapted to be smaller when the catheter is introduced into the arteries and/or veins of the user. By having a first cross-sectional diameter that is smaller than the second cross-sectional diameter, the fluid guide element may have a diameter that is close to or equal to the cross-sectional diameter of the catheter, so that the catheter can enter through a small opening in the skin surface, and that the maximal diameter of the catheter during insertion is as small as possible. By having a second cross-sectional diameter that is larger than the first cross-sectional diameter, the fluid guide element may in its insertion state be formed so that the blood flow past the catheter is not impeded by the fluid guide element in its insertion state, but when the fluid guide element is in its measurement state, the fluid guide element is capable of increasing its area in the area that faces the flow direction of the blood, in order to intersect the blood, and change the direction of the blood flow towards the first opening. Thus, the fluid guide element may have a collapsed state, which may be the insertion state, and may have an expanded state, which may be the measurement state.

In one or more exemplary embodiments, the fluid guide element may have a concave distal part facing the distal end of the catheter tube, where the concave part is configured to guide at least part of the surrounding arterial flow into the first opening. The concave part of the fluid guide element may be a curved part, where the bottom of the curve faces away from the flow direction of the blood, so that the curved part of the fluid guide element may gradually change the direction of the blood flow towards the first opening. The concave part of the fluid guide element may create a volume, between the edges of the concave part, where blood may be collected, and the flow of the blood in the artery may force the blood gathered in the volume (guide volume) towards the first opening of the catheter. The first opening may abut the end of the fluid guide element that is closer to the catheter tube, so that the blood flow may push the blood into the first opening.

In one or more exemplary embodiments, the concave distal part may be connected to a part of the catheter tube that is between a proximal end of the catheter and the first opening. The proximal part of the catheter may have a tip, as disclosed in the present disclosure. The first opening may be positioned at a predetermined distance from the proximal part, where the concave part of the fluid guide element may be positioned in an area of the catheter that is between the proximal part and the longitudinal position of the first opening. This means that the flow of blood in the artery may be adapted to flow along the outer surface of the catheter, where the blood may pass the first opening, and subsequently the blood will intersect the concave distal part. Thus the distal part of the flow guide may be adapted to guide a part of the blood flow in a direction that is different from the flow of blood through the artery, and where the opening may be prior to the flow guide element, a part of the blood flow may be forced in a direction that is opposed to the direction of flow through the artery. Thus, the distal concave part of the fluid guide element may be adapted to force the flow of blood into the opening and the lumen in a direction that is opposite to the flow of blood inside the artery. I.e. the guide element may be configured to intercept and change the flow direction of at least part of the blood flow inside the artery.

The fluid guide element may e.g. be an umbrella shaped or a V-shaped part, where the peripheral edge of the fluid guide element faces is closer to the distal part of the catheter than the central part of the fluid guide element, creating a fluid guide volume between the peripheral edge and the catheter tube (in a radial direction).

In one or more exemplary embodiments, the fluid guide element may be an inflatable balloon connected to the conversion element of the catheter. The fluid guide element may be in the form of a balloon that has a predefined shape when inflated, where the predefined shape may be adapted to guide the flow of blood towards the first opening. The conversion element may be means adapted to inflate the balloon, where the inflation of the balloon may be done by forcing a fluid through a channel, where the channel is in fluid communication with the balloon. The inflation of the balloon may provide a pressure inside the balloon that is larger than the pressure inside the artery, so that the shape of the balloon may be maintained during measurement. The positioning and/or the shape of the balloon may be in accordance with the disclosed shapes of the fluid guide element, where the distal part of the balloon may have a shape that may guide at least part of the blood flow towards the first opening.

In one or more exemplary embodiments, the conversion element may have a second inner lumen providing fluid communication along the longitudinal axis of the catheter tube, where the second inner lumen may be in fluid communication with the fluid guide element, to allow the selectively conversion of the fluid guide element from the insertion state to the measuring state, and vice versa. The second inner lumen may be parallel to the first inner lumen, where a fluid or a mechanical actuator may be introduced into the second lumen to manipulate the fluid guide element from its insertion state to its measurement state, and vice versa. The second lumen may extend along the longitudinal length of the catheter in similar manner to the first lumen, where a distal end of the second lumen may extend from the distal end of the catheter, and a the proximal end of the second lumen may have an opening from inside the catheter tube through the wall of the catheter tube.

Alternatively, a balloon element may close off the proximal end of the second lumen, so that a fluid inside the second lumen can inflate the balloon.

In one or more exemplary embodiments, the first inner lumen of the catheter tube may be in fluid communication with the flowmeter via an arterial flow tube. The arterial flow tube may be any kind of tube which may be in fluid communication with the first inner lumen and the flowmeter. The arterial flow tube may e.g. be a tube which is connected to a distal end of the catheter, and may extend from the distal end of the catheter and out of the body. The arterial flow tube may have a connector at its distal end, where the connected arterial flow tube may be in fluid communication with the flowmeter. The arterial tube may have two or more lumens, where each lumen may connect to one specific (first, second or third) lumen in of the catheter.

In one or more exemplary embodiments, the arterial flow tube may have a proximal end connected to the first inner lumen of the catheter and a distal end connected to the flowmeter. Thus, the arterial flow tube may be an extension of the first inner lumen of the catheter, and may communicate blood flow from the first inner lumen to the flowmeter.

In one or more exemplary embodiments, the catheter tube may comprise a third inner lumen extending along the longitudinal axis of the catheter tube, where third inner lumen may provide a return conduit for arterial flow from the flowmeter, and may be in fluid communication with a second opening (output opening) positioned on the catheter tube. Thus, the third inner lumen of the catheter tube may be utilized to provide a return fluid communication for blood that is to be extracted from the artery, so that the user will not lose a significant amount of blood during the flow measurement of the arterial blood.

In one or more exemplary embodiments, the second opening may be positioned proximally to the first opening and/or the fluid guide element. Thus, the second opening is positioned downstream to the first opening and/or the fluid guide element, so that the return of the blood occurs after the blood has been extracted from the artery and/or vein. This means that the return of the blood will have a minimized effect on the blood flow measurements that are occurring upstream from the second opening.

In one or more exemplary embodiments, the second opening may be positioned on a terminal proximal end of the catheter tube, optionally where a central axis of the second opening is coaxial with the longitudinal axis of the catheter tube. Thus, the second opening may be arranged on the proximal tip of the catheter, and the third lumen may be arranged in a central area of the catheter, so that the second opening may be return the extracted blood in along the longitudinal axis of the catheter, and the fluid communication out of the third lumen occurs somewhat in a parallel with the flow of blood inside the artery. The third inner lumen may e.g. be positioned between a first lumen and a second lumen, where the third inner lumen is positioned centrally, and the first and the second lumens are positioned on opposing sides of the third lumen.

In one or more exemplary embodiments, a central axis of the first opening may be perpendicular to the longitudinal axis of the catheter tube. The central axis of the first opening may be an axis that extends at a centre of a plane that is the first opening. Thus, the central axis is normal to the plane of the opening. The flow guide element may be adapted to alter the direction of at least part of the flow of the blood through the artery, and where the flow guide may be adapted to change the direction of flow of the blood prior to it entering the first lumen and the first opening, and where the flow guide may change the direction of flow to approximately 90 degrees, for the blood to enter the first opening. After the blood has entered the first opening, the direction of flow of blood inside the lumen is approximately opposite to the direction of flow inside the artery.

In one or more exemplary embodiments, the first inner lumen, second inner lumen and/or third inner lumen are separated from each other. The separation of the first, second and/or third inner lumen means that it may be possible to provide fluid communication to different parts of the catheter via the lumens. The second lumen may provide fluid communication to the fluid guide element, where the first and third lumens may be adapted to provide an output blood flow and an input blood flow, respectively. Thus, by isolating the fluid communication between the lumens, it is possible to provide reliable measurements, without the flow being interrupted by other functionalities of the catheter.

In one or more exemplary embodiments, an input part of the flowmeter may be in fluid communication with the first lumen of the catheter tube, and/or an output part of the flowmeter is in fluid communication with a third inner lumen of the catheter tube. Thus, the catheter and the flowmeter create a closed system, where blood is drawn from the artery via the first lumen, the flow of the blood is measured, and the blood is returned to the artery via the second opening and the third lumen. The closed system may ensure that the blood does not oxidize during flow measurements, or come into contact with the surrounding environment, which means that the blood may be reintroduced into the body after the flow measurement.

In one or more exemplary embodiments, the distal end of the catheter tube may comprise a first connector for the first internal lumen, a second connector for the second internal lumen and/or a third connector for the third internal lumen. The first, second and/or third connectors may allow each lumen to be separately connected to a predefined source of fluid communication with each lumen. Alternatively, one or more of the lumens may have joint connectors, where each lumen has a fluid communication pathway, but where the connector secures the connection between the catheter lumen and a fluid communication pathway, such as a flow tube.

The present disclosure may also relate to a method of measuring arterial flow in an artery, the method comprising: inserting a catheter having a longitudinal axis, a proximal end and a distal end, a first inner lumen providing fluid communication along the longitudinal axis of the catheter tube, a first opening (input opening) configured to provide fluid communication from the outside of the catheter tube and into the first inner lumen through a side wall of the catheter tube, a fluid guide element having an insertion state where the fluid guide element has a first cross-sectional diameter, and in a measuring state where the fluid guide element has a second cross-sectional diameter, where the fluid guide element is configured to guide at least part of an arterial flow surrounding the fluid guide element into the first opening, a conversion element, where the conversion element is connected to the fluid guide element allowing a user to selectively transform the fluid guide element from an insertion state to a measuring state, and vice versa; converting the fluid guide element to a measuring state where the fluid guide element guides the arterial blood into the first opening; extracting arterial blood along the first inner lumen and into a flowmeter, where the flowmeter measures the flow rate of the blood entering the flowmeter.

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### BRIEF DESCRIPTION OF THE DRAWINGS

The following is an explanation of exemplary embodiments with reference to the drawings, in which

FIG. 1 is a perspective frontal view of a catheter,

FIG. 2 is a perspective rear view of a catheter,

FIGS. 3a and 3b are side views of a catheter in an measurement state and a insertion state, respectively,

FIG. 4 is a side sectional view of a catheter, and

FIG. 5 is a schematic view of a arterial flow measurement system.

### DETAILED DESCRIPTION

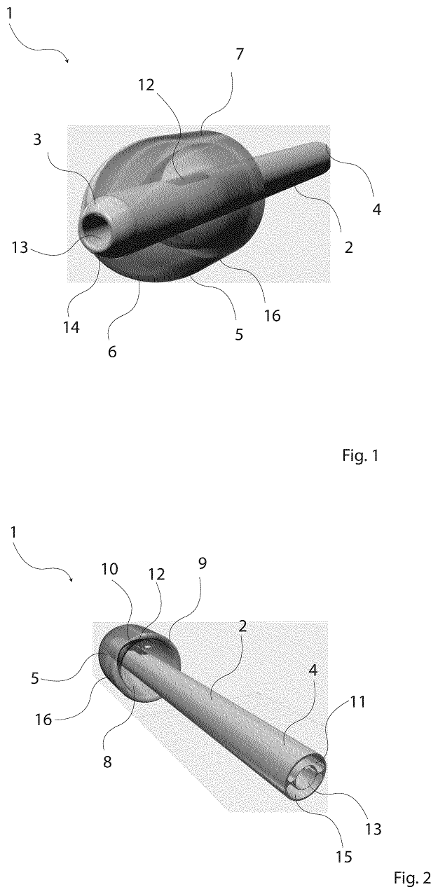

FIG. 1 and FIG. 2 show a front perspective view and a rear perspective view of a catheter 1, respectively. The catheter 1 comprises a catheter tube 2 having a proximal end 3 and a distal end 4. The catheter 1 may comprise a fluid guide 5, where the fluid guide 5 extends around the circumference of the catheter tube 2, and provides an area of a catheter 1, i.e. the fluid guide, where the catheter 1 has an increased diameter. The fluid guide 5 has a proximal part 6 and a distal part 7, where the proximal part 6 of the fluid guide faces the proximal end 3 of the catheter tube 2 and the distal part 7 of the fluid guide 5 faces the proximal end 3 of the catheter tube 2. The distal part 7 of the fluid guide 5 may have a collection volume 8, where the collection volume 8 may be a concave part, where a distal peripheral edge 9 of the fluid guide extends in a direction closer to the distal end 3 of the catheter tube 2 than an inner radial part 10, which abuts the catheter tube 2. The collection volume 8 of the fluid guide 5 is configured to face the flow direction of arterial blood, so that the blood is adapted to be captured in the collection volume 8.

The catheter tube 2 may comprise a first lumen 11, which extends along the length of the catheter tube 2, where the first lumen 11 terminates in a first opening 12, which abuts the inner radial part 10 of the fluid guide 5 in a distal direction, so that the blood that is collected in the collecting volume 8, may be diverted into the first opening 12, allowing the blood to be directed out of the artery, via the first opening 12 and along the first lumen 11 in a proximal direction. The first lumen 11, may be in fluid connection with an input of a flowmeter (not shown) to measure the flow of blood through the first lumen 11.

The catheter tube 2, may comprise a third lumen 13, which extends along the length of the catheter tube 2, where the third lumen 13 terminates in the proximal end 3 of the catheter tube 2, in the form of a second opening 14. The third lumen 13 may be a fluid communication to an output of a flowmeter (not shown) inside the catheter tube, where the third lumen 13 may be utilized to return blood back to an artery after the blood has been diverted out of an artery, where the blood exits the second opening 14 in a direction that is in parallel to the blood flow inside the artery.

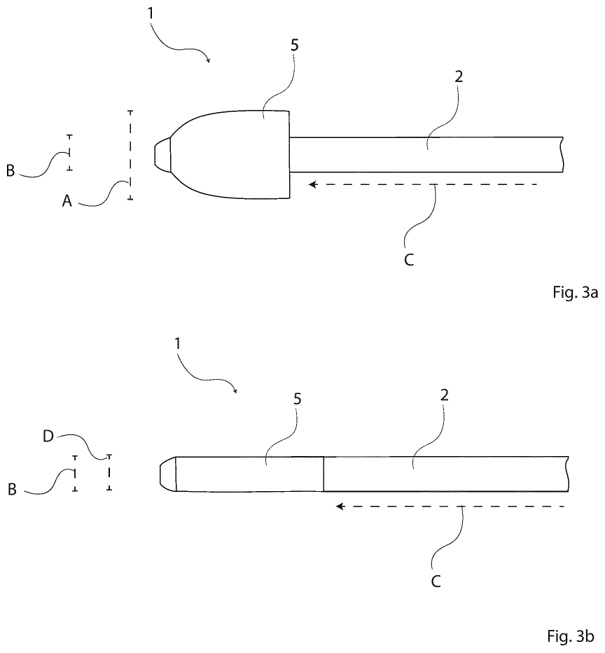

The catheter tube 2, may comprise a second lumen 15, where the second lumen 15 may be in fluid communication with the fluid guide 5, where the second lumen 15 may be utilized to accommodate a conversion element that is capable of providing means of transitioning the fluid guide 5 from an insertion state (shown in FIG. 3b), to a measurement state (shown in FIG. 3a). The fluid guide 5 may be a shaped balloon 16, where the shape of the balloon in a measurement state is the form shown in the present disclosure, allowing the fluid guide 5 to guide the flow of fluid into the first opening 12.

FIG. 3a shows the catheter 1, where the fluid guide 5 is in a measurement state, i.e. in an expanded state. When the fluid guide 5 is in a measurement state, the cross-sectional diameter A of the fluid guide 5 is larger than the cross-sectional diameter B of the catheter tube 2, where the increased diameter means that flow of blood in a direction C can be collected inside the collecting volume 8, and guided towards the first opening (not shown).

FIG. 3b shows the catheter 1, where the fluid guide 5 is in a insertion state, i.e. in a collapsed state. When the fluid guide 5 is in an insertion state, the cross-sectional diameter D of the fluid guide 5 is substantially equal to the cross-sectional diameter B of the catheter tube, where the collapsed fluid guide 5 means that the fluid guide does not interfere with the flow of blood in a direction C more than the catheter tube 2 does. The insertion state of the fluid guide 5 may also be utilized, when the catheter 1 is to be extracted from an artery.

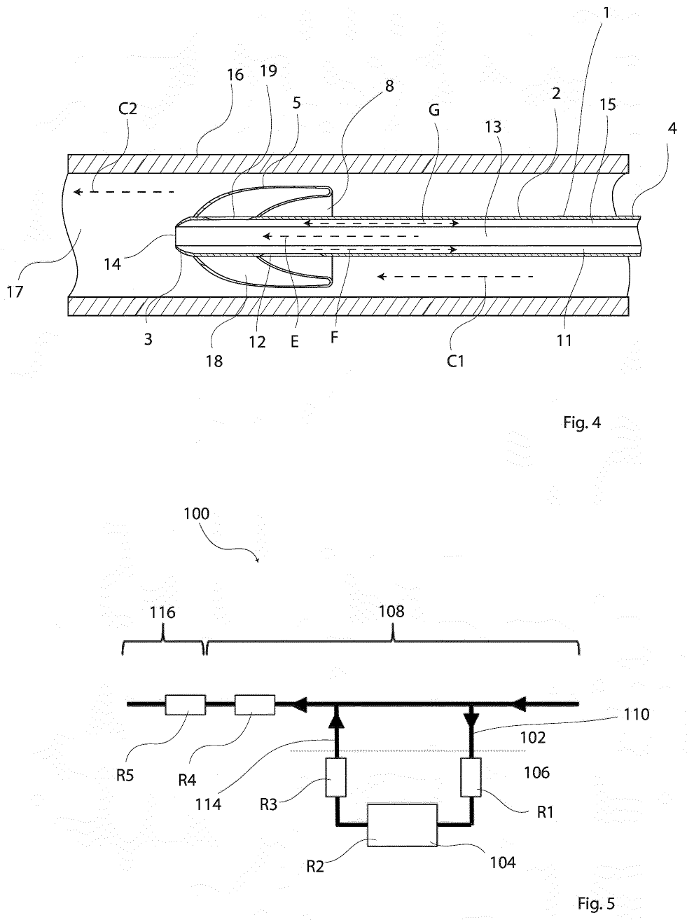

FIG. 4 shows a cross-sectional schematic view of a catheter 1 which is introduced into an artery 16, where the fluid guide 5 is in its measurement state, i.e. in an expanded state, where the cross-sectional diameter of the fluid guide 5 is larger than the cross-sectional diameter of the catheter tube 2. The flow of blood inside 17 the artery may be seen as the distal flow C1 and the proximal flow C2, where the distal flow C1 may be seen as the flow which the fluid guide can intersect, while the proximal flow C2 may be seen as the flow which passes the fluid guide 5 and also the blood flow which exits the second opening 14, in a direction E, where the direction E may be the same direction as the flow directions C1 and C2.

The blood flow C1 may be collected inside the collecting volume 8 of the fluid guide 5, where the blood may be guided into the first opening 12, where the flow of blood may be seen as reversing in a direction F inside the first lumen 11, which is an opposite direction to the direction C1, where the first lumen may be fluid communication with a flowmeter (not shown) which is capable of measuring the flow of blood inside the first lumen 11. When the flowmeter (not shown) has measured the blood flow, the blood may be returned via the third lumen 13, and exits the catheter at its proximal end 3 through the second opening 14, which returns the blood into the inner volume 17 of the artery 16.

The catheter 1 also comprises a second lumen 15, where the second lumen 15 is in fluid communication with an inner volume 18 of the fluid guide 5, where the fluid communication may e.g. be through a third opening 19, which allows fluids to pass from the second lumen 15 and into the inner volume 18 of the fluid guide 5. The fluid guide 5 in this example may be an inflatable balloon, which may be inflated and deflated by communicating a fluid in the both a proximal direction and a distal direction, as shown with arrow G, to selectively inflate and/or deflate the inner volume 18 of the fluid guide 5, to transform the fluid guide 5 from an insertion state to a measurement state (as shown in FIGS. 3a and 3b).

FIG. 5 shows a schematic diagram of a system 100 for measuring flow in an artery 108 and/or a vein 108 inside the body 102, where the flowmeter 104 may be positioned outside 106 the body. A part of the blood flow 110 may be diverted from the artery 108 and into a flowmeter 104, where the diverted blood flow 110 may be returned 114 back into the artery 108, when the flow of the diverted blood flow 110 has been measured by the flowmeter.

The calculations regarding the measured flow in the system 100 may be influenced by a resistance in the system, where resistance may be divided into resistance R1, which is the resistance in the tubes diverting the blood from the artery 108, resistance R2 which is the resistance in the flowmeter, resistance R3 which is the resistance in the fluid communication leading the blood flow back into the artery 108, resistance R4 which may be the resistance of inside the artery 108 due to the insertion of the catheter and/or the resistance R5 which may be the resistance in microcirculation 116 which is in fluid communication with the artery. If the resistances in the system 100 may be measured or calculated the flow measured in the flowmeter may be mathematically transformed to represent the actual flow in the artery. The shape and size of the fluid guide and the catheter will influence the measurements, and by performing tests and experiments the correlation between the fluid guide and the collected flow of blood may be established, to assist in the calculation of the blood flow through the artery.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

1. An arterial flow measurement system comprising:
a catheter tube being adapted to be inserted into an artery, the catheter tube having longitudinal axis, a proximal end and a distal end,
a first inner lumen providing fluid communication along the longitudinal axis of the catheter tube,
a first opening configured to provide fluid communication from the outside of the catheter tube and into the first inner lumen through a side wall of the catheter tube,
a fluid guide element configured to guide at least part of an arterial flow surrounding the fluid guide element into the first opening,
a flowmeter configured to measure the flow of a fluid, wherein the flowmeter is an outside-the-body flowmeter, and where the flowmeter is in fluid communication with the first inner lumen of the catheter tube; and
the catheter tube comprising a third inner lumen extending along the longitudinal axis of the catheter tube, where the third inner lumen provides a return conduit for arterial flow from the flowmeter, and is in fluid communication with a second opening (output opening) positioned on the catheter tube.

2. An arterial flow measurement system in accordance with claim 1, wherein the fluid guide element is positioned between a proximal tip of the catheter and the first opening.

3. An arterial flow measurement system in accordance with claim 1, wherein the first cross-sectional diameter is smaller than the second cross-sectional diameter.

4. An arterial flow measurement system in accordance with claim 1, wherein the fluid guide element has a concave distal part facing the distal end of the catheter tube, where the concave part is configured to guide at least part of the surrounding arterial flow into the first opening.

5. An arterial flow measurement system in accordance with claim 3, wherein the concave distal part is connected to a part of the catheter tube that is between a proximal end of the catheter and the first opening.

6. An arterial flow measurement system in accordance with claim 17, wherein the fluid guide element is an inflatable balloon connected to the conversion element of the catheter.

7. An arterial flow measurement system in accordance with claim 17, wherein the conversion element has a second inner lumen providing fluid communication along the longitudinal axis of the catheter tube, where the second inner lumen is in fluid communication with the fluid guide element.

8. An arterial flow measurement system in accordance with claim 1, wherein the first inner lumen of the catheter tube is in fluid communication with the flowmeter via an arterial flow tube, where the arterial flow tube has a proximal end connected to the first inner lumen of the catheter and a distal end connected to the flowmeter.

9. An arterial flow measurement system in accordance with claim 1, wherein the second opening is positioned proximally to the first opening and/or the fluid guide element.

10. An arterial flow measurement system in accordance with claim 1, wherein the second opening is positioned on a terminal proximal end of the catheter tube, optionally where a central axis of the second opening is coaxial with the longitudinal axis of the catheter tube.

11. An arterial flow measurement system in accordance with claim 1, wherein a central axis of the first opening is perpendicular to the longitudinal axis of the catheter tube.

12. An arterial flow measurement system in accordance with claim 1, wherein the first inner lumen, second inner lumen and/or third inner lumen are separated from each other.

13. An arterial flow measurement system in accordance with claim 1, wherein in input part of the flowmeter is in fluid communication with the first lumen of the catheter tube, and/or an output part of the flowmeter is in fluid communication with the third inner lumen.

14. An arterial flow measurement system in accordance with claim 1, wherein the distal end of the catheter tube comprises a first connector for the first internal lumen, a second connector for the second internal lumen and/or a third connector for the third internal lumen.

15. (canceled)

16. (canceled)

17. An arterial flow measurement system in accordance with claim 1, further comprising a conversion element, where the conversion element is connected to the fluid guide element allowing a user to selectively transform the fluid guide element from an insertion state to a measuring state, and vice versa.

18. An arterial flow measurement system in accordance with claim 1, wherein the fluid guide element has an insertion state where the fluid guide element has a first cross-sectional diameter and in a measuring state where the fluid guide element has a second cross-sectional diameter.

19. An arterial flow measurement system in accordance with claim 18 wherein:
the arterial flow measurement system comprises a conversion element, where the conversion element is connected to the fluid guide element allowing a user to selectively transform the fluid guide element from an insertion state to a measuring state, and vice versa; and
the conversion element has a second inner lumen providing fluid communication along the longitudinal axis of the catheter tube, where the second inner lumen is in fluid communication with the fluid guide element, to allow the selectively conversion of the fluid guide element from the insertion state to the measuring state, and vice versa.

20. A method of measuring arterial flow in an artery, the method comprising:
inserting a catheter tube having a longitudinal axis, a proximal end and a distal end, a first inner lumen providing fluid communication along the longitudinal axis of the catheter tube, a first opening (input opening) configured to provide fluid communication from the outside of the catheter tube and into the first inner lumen through a side wall of the catheter tube, and a fluid guide element configured to guide at least part of an arterial flow surrounding the fluid guide element into the first opening; and
extracting arterial blood along the first inner lumen and into a flowmeter, where the flowmeter measures the flow rate of the blood entering the flowmeter.

21. A method in accordance with claim 20, wherein the fluid guide element comprises an insertion state where the fluid guide element has a first cross-sectional diameter, and a measuring state where the fluid guide element has a second cross-sectional diameter.

22. A method in accordance with claim 21, wherein catheter tube comprises a conversion element, where the conversion element is connected to the fluid guide element allowing a user to selectively transform the fluid guide element from the insertion state to the measuring state, and vice versa, wherein the method further comprises:
converting the fluid guide element to a measuring state where the fluid guide element guides the arterial blood into the first opening.
